(19) United States
(12) Patent Application Publication
(10) Pub. No.: US 2025/0339489 A1
(43) Pub. Date: Nov. 6, 2025

(54) COMPOSITION FOR SUPPRESSING HAIR LOSS OR PROMOTING HAIR GROWTH CONTAINING PEPTIDE

(71) Applicant: AMOREPACIFIC CORPORATION, Seoul (KR)

(72) Inventors: Jinoh CHUNG, Yongin-si (KR); Ahreum KIM, Yongin-si (KR); Hyewon MOK, Yongin-si (KR); Jonghee SOHN, Yongin-si (KR); Wanki KIM, Yongin-si (KR)

(73) Assignee: AMOREPACIFIC CORPORATION, Seoul (KR)

(21) Appl. No.: 19/173,342

(22) Filed: Apr. 8, 2025

(30) Foreign Application Priority Data
May 3, 2024 (KR) 10-2024-0059137
May 3, 2024 (KR) 10-2024-0059138
Jun. 26, 2024 (KR) 10-2024-0083424
Jun. 27, 2024 (KR) 10-2024-0084248
Aug. 28, 2024 (KR) 10-2024-0115702

(51) Int. Cl. A61K 38/06; A61K 8/64; A61P 17/14; A61Q 7/00

(52) U.S. Cl. CPC A61K 38/06; A61K 8/64; A61P 17/14; A61Q 7/00

(57) ABSTRACT

A method for suppressing hair loss or promoting hair growth including administering particular peptides to a subject in need thereof is disclosed. Specifically, the disclosed method can effectively suppress hair loss and promote hair growth by promoting or activating the growth or proliferation of hair dermal papilla cells. The method includes administering peptides formulated into various types of compositions.

### CROSS-REFERENCE TO RELATED APPLICATIONS

The present application claims priority to Korean Patent Application Nos. 10-2024-0059137; 10-2024-0059138; 10-2024-0083424; 10-2024-0084248; and 10-2024-0115702 filed May 3, 2024; May 3, 2024; Jun. 26, 2024; Jun. 27, 2024; and Aug. 28, 2024, respectively, the entire contents of which are hereby incorporated by this reference.

### BACKGROUND

### Field of the Invention

The present disclosure relates to a composition for suppressing hair loss or promoting hair growth, including a particular peptide.

### Description of the Related Art

Human hair is primarily responsible for a defensive function to protect the scalp from external stimuli such as ultraviolet rays and an aesthetic function to express the external image of an individual. However, in modern society, hair loss is becoming more common due to natural factors such as environmental pollution and strong ultraviolet rays, as well as physiological factors such as stress and hormonal imbalance, and unlike in the past when hair loss was limited to adult men, it is now occurring in people of all ages and genders, so that there is an urgent need for materials for preventing and treating hair loss.

Hair follicles, which are physiological organs that produce hair in the body, repeat a hair cycle that is divided into the anagen phase when is produced and the produced hair actively grows, the catagen phase when the hair degenerates, the telogen phase when the hair is maintained until it falls out, and the exogen phase when which hair loss occurs, during postnatal development, and are involved in the growth, maintenance, and shedding of hair. Many studies have revealed that the activity of the hair follicles is caused by hair dermal papilla cells, and in particular, the proliferation and differentiation of hair dermal papilla cells are primarily involved in the progression of the hair growth cycle and hair formation. During the anagen phase when hair actively grows, the active proliferation and differentiation of hair dermal papilla cells occurs, and during the catagen, telogen and exogen phases when hair growth stops and hair loss occurs, these cells die. Therefore, since hair growth and hair loss are closely associated with the proliferation and death of hair dermal papilla cells, lengthening the anagen phase by inducing the proliferation of these cells, or suppressing cell death and shortening the catagen, telogen, and exogen phases would be a method for improving and treating hair loss.

Alopecia refers to a condition in which more than 100 hairs fall out per day and the number of hairs in the anagen phase decreases and the number of hairs in the catagen or telogen phase increases, and as hair loss progresses, the proportion of hair in the telogen phase increases rapidly and the proportion of hair in the anagen phase decreases accordingly. Hair loss may generally be classified into hereditary androgenetic alopecia (baldness), alopecia areata, tinea capitis caused by fungal infection, telogen effluvium, trichotillomania, hair production disorder, and the like. Among them, the most common types of alopecia include baldness (male pattern alopecia), female pattern alopecia, alopecia areata, telogen effluvium, and the like. There are many causes of hair loss. Genetic factors and the male hormone androgen are considered to be important factors in the occurrence of baldness, and it is assumed that some female pattern hair loss occurs in a similar manner to male pattern hair loss, but there are differences in clinical aspects. It is assumed that alopecia areata is caused by an autoimmune disease. Telogen effluvium is a temporary hair loss that occurs after severe physical or mental stress such as endocrine disorders, nutritional deficiencies, drugs, childbirth, fever, and surgery, and it occurs when some hair does not complete the entire growth period and transitions into the telogen phase, falling out.

### SUMMARY OF THE INVENTION

An object of the present disclosure is to provide a composition that exhibits the excellent effect of suppressing hair loss or promoting hair growth by promoting or activating the growth or proliferation of hair dermal papilla cells.

To achieve the aforementioned object, an embodiment of the present invention provides a method for suppressing hair loss or promoting hair growth, the method comprising: administering an effective amount of a peptide including an amino acid sequence of Gly-Pro-Lys (GPK), an amino acid sequence of Gly-Pro-Ser (GPS), an amino acid sequence of Gly-Pro-Ala (GPA), an amino acid sequence of Gly-Gln-Lys (GQK), or an amino acid sequence of Pro-Hyp (PO); or a salt thereof to a subject in need thereof.

The method according to the present disclosure can effectively suppress hair loss and promote hair growth by promoting or activating the growth or proliferation of hair dermal papilla cells.

### BRIEF DESCRIPTION OF THE DRAWINGS

FIG. 1 illustrates the results of confirming the effect of GPK peptide in activating the proliferation of hair dermal papilla cells.

FIG. 2 illustrates the results of confirming the effect of GPS peptide in activating the proliferation of hair dermal papilla cells.

FIG. 3 illustrates the results of confirming the effect of GPA peptide in activating the proliferation of hair dermal papilla cells.

FIG. 4 illustrates the results of confirming the effect of GQK peptide in activating the proliferation of hair dermal papilla cells.

FIG. 5 illustrates the results of confirming the effect of PO peptide in activating the proliferation of hair dermal papilla cells.

### DETAILED DESCRIPTION OF THE INVENTION

Hereinafter, the present invention will be described in detail.

In one aspect, the present invention may relate to a method for suppressing hair loss or promoting hair growth, the method comprising administering an effective amount of a peptide including an amino acid sequence of Gly-Pro-Lys (GPK) or a salt thereof to a subject in need thereof.

In one aspect, the present invention may relate to a method for suppressing hair loss or promoting hair growth, the method comprising administering an effective amount of a peptide including an amino acid sequence of Gly-Pro-Ser (GPS) or a salt thereof to a subject in need thereof.

In one aspect, the present invention may relate to a method for suppressing hair loss or promoting hair growth, the method comprising administering an effective amount of a peptide including an amino acid sequence of Gly-Pro-Ala (GPA) or a salt thereof to a subject in need thereof.

In one aspect, the present invention may relate to a method for suppressing hair loss or promoting hair growth, the method comprising administering an effective amount of a peptide including an amino acid sequence of Gly-Gln-Lys (GQK) or a salt thereof to a subject in need thereof.

In one aspect, the present invention may relate to a method for suppressing hair loss or promoting hair growth, the method comprising administering an effective amount of a peptide including an amino acid sequence of Pro-Hyp (PO) or a salt thereof to a subject in need thereof.

In one aspect, the present invention may relate to a method for suppressing hair loss or promoting hair growth, the method comprising administering an effective amount of a peptide including an amino acid sequence of Gly-Pro-Thr (GPT) or a salt thereof to a subject in need thereof.

In one aspect, the present invention may relate to a method for suppressing hair loss or promoting hair growth, the method comprising administering an effective amount of a peptide including an amino acid sequence of Arg-Lys (RK) or a salt thereof to a subject in need thereof.

In one aspect, the present invention may relate to a method for suppressing hair loss or promoting hair growth, the method comprising administering an effective amount of a peptide including an amino acid sequence of Pro-Gly-Glu (PGE) or a salt thereof to a subject in need thereof.

In one aspect, the present invention may relate to a composition for suppressing hair loss or promoting hair growth, comprising a peptide including an amino acid sequence of Gly-Pro-Lys (GPK) or a salt thereof as an active ingredient.

In one aspect, the present invention may relate to a composition for suppressing hair loss or promoting hair growth, comprising a peptide including an amino acid sequence of Gly-Pro-Ser (GPS) or a salt thereof as an active ingredient.

In one aspect, the present invention may relate to a composition for suppressing hair loss or promoting hair growth, comprising a peptide including an amino acid sequence of Gly-Pro-Ala (GPA) or a salt thereof as an active ingredient.

In one aspect, the present invention may relate to a composition for suppressing hair loss or promoting hair growth, comprising a peptide including an amino acid sequence of Gly-Gln-Lys (GQK) or a salt thereof as an active ingredient.

In one aspect, the present invention may relate to a composition for suppressing hair loss or promoting hair growth, comprising a peptide including an amino acid sequence of Pro-Hyp (PO) or a salt thereof as an active ingredient.

In one aspect, the present invention may relate to a composition for suppressing hair loss or promoting hair growth, comprising a peptide including an amino acid sequence of Gly-Pro-Thr (GPT) or a salt thereof as an active ingredient.

In one aspect, the present invention may relate to a composition for suppressing hair loss or promoting hair growth, comprising a peptide including an amino acid sequence of Arg-Lys (RK) or a salt thereof as an active ingredient.

In one aspect, the present invention may relate to a composition for suppressing hair loss or promoting hair growth, comprising a peptide including an amino acid sequence of Pro-Gly-Glu (PGE) or a salt thereof as an active ingredient.

Amino acids referred to as abbreviations in the present specification were described according to IUPAC-IUB nomenclature as follows:

Glycine: G, Proline: P, Lysine: K, Serine: S, Alanine: A, Glutamine: Q, Hydroxyproline: O, Threonine: T, Arginine: R, and Glutamic Acid: E

In an embodiment, the peptide may have a proportion of GPK, GPS, GPA, GQK, PO, GPT, RK, or PGE amino acid of 50% or more, 60% or more, 70% or more, 80% or more, or 90% or more, preferably 95% or more, and most preferably 100%.

In an embodiment, the peptide may be a GPK, GPS, GPA, GQK, PO, GPT, RK, or PGE peptide. That is, the peptide may be a peptide composed of an amino acid sequence of Gly-Pro-Lys, an amino acid sequence of Gly-Pro-Ser, an amino acid sequence of Gly-Pro-Ala, an amino acid sequence of Gly-Gln-Lys, an amino acid sequence of Pro-Hyp, an amino acid sequence of Gly-Pro-Thr, an amino acid sequence of Arg-Lys, or an amino acid sequence of Pro-Gly-Glu.

In the present specification, even though a peptide is described as “a peptide composed of a specific amino acid sequence”, a case where the peptide has the same or equivalent activity as a peptide composed of the corresponding amino acid sequence does not exclude meaningless addition of sequences before or after the corresponding amino acid sequence, or naturally occurring mutations, or silent mutations thereof, and it is self-evident that a case where the peptide has such addition of sequences or mutations also falls within the scope of the present application.

In an embodiment, the peptide may be prepared by chemical synthesis methods, standard synthesis methods, enzymatic or base or acid proteolytic methods, or any other methods known in the art.

In the present specification, the “peptide salt” may be a pharmaceutically or cosmetically acceptable salt. Specifically, the peptide salt may be formed by adding an acid to the peptide, for example, by adding an inorganic acid (for example: hydrochloric acid, hydrobromic acid, phosphoric acid, nitric acid, sulfuric acid, and the like), an organic carboxylic acid (for example: acetic acid, haloacetic acid such as trifluoroacetic acid, propionic acid, maleic acid, succinic acid, malic acid, citric acid, tartaric acid, and salicylic acid), an acidic sugar (glucuronic acid, galacturonic acid, gluconic acid, and ascorbic acid), an acidic polysaccharide (for example: hyaluronic acid, chondroitin sulfate, and alginic acid), an organic sulfonic acid including a sulfonic acid sugar ester such as chondroitin sulfate (for example: methanesulfonic acid and p-toluenesulfonic acid), and the like to the peptide.

In an embodiment, the peptide or salt thereof may be formulated into the form of a composition.

In one embodiment, the peptide or salt thereof may be included in the composition at a concentration of 0.001 to 200 M.

For example, the concentration of the peptide or salt thereof in the composition may be 0.001 UM or more, 0.005 μM or more, 0.01 μM or more, 0.05 μM or more, 0.1 μM or more, 0.5 μM or more, 1 μM or more, 5 μM or more, 10 μM or more, 15 μM or more, 20 μM or more, 25 μM or more, 30 μM or more, 35 μM or more, 40 μM or more, 45 μM or more, 50 μM or more, 55 μM or more, 60 μM or more, 65 μM or more, 70 μM or more, 75 μM or more, 80 μM or more, 85 μM or more, 90 μM or more, 95 μM or more, 100 μM or more, 110 μM or more, 120 μM or more, 130 μM or more, 140 μM or more, 150 μM or more, 160 μM or more, 170 μM or more, 180 μM or more, or 190 μM or more, and in addition, the concentration of the peptide or salt thereof in the composition may be 200 μM or less, 190 μM or less, 180 μM or less, 170 μM or less, 160 μM or less, 150 μM or less, 140 μM or less, 130 μM or less, 120 μM or less, 110 μM or less, 100 μM or less, 95 μM or less, 90 μM or less, 85 μM or less, 80 μM or less, 75 μM or less, 70 μM or less, 65 μM or less, 60 μM or less, 55 μM or less, 50 μM or less, 45 μM or less, 40 μM or less, 35 μM or less, 30 μM or less, 25 μM or less, 20 μM or less, 15 μM or less, 10 μM or less, 5 μM or less, 1 μM or less, 0.5 μM or less, 0.1 μM or less, 0.05 μM or less, 0.01 μM or less, or 0.005 μM or less.

In an embodiment, cells may be treated with the peptide or salt thereof at a concentration of 0.1 to 10 μM.

For example, cells may be treated with the peptide or salt thereof at a concentration of 0.1 μM or more, 0.2 μM or more, 0.3 μM or more, 0.4 μM or more, 0.5 μM or more, 0.6 μM or more, 0.7 μM or more, 0.8 μM or more, 0.9 μM or more, 1 μM or more, 2 μM or more, 3 μM or more, 4 μM or more, 5 μM or more, 6 μM or more, 7 μM or more, 8 μM or more, or 9 μM or more, and in addition, cells may be treated with the peptide or salt thereof at a concentration of 10 UM or less, 9 μM or less, 8 μM or less, 7 μM or less, 6 μM or less, 5 μM or less, 4 μM or less, 3 μM or less, 2 μM or less, 1 μM or less, 0.9 μM or less, 0.8 μM or less, 0.7 μM or less, 0.6 μM or less, 0.5 μM or less, 0.4 μM or less, 0.3 μM or less, or 0.2 μM or less.

In an embodiment, the cells may be hair dermal papilla cells.

In an embodiment, the peptide or salt thereof may promote or activate the growth or proliferation of hair dermal papilla cells.

In an embodiment, the composition may be a pharmaceutical composition for preventing or treating hair loss.

As used herein, the “prevention” refers to reducing the risk of developing a disease or disorder, and refers to any action that inhibits or delays the onset of a disease by preventing one or more clinical symptoms of the disease from progressing in a subject who has been exposed to the disease or is susceptible to the disease, but has not yet developed the disease or exhibited symptoms of the disease.

As used herein, the “treatment” refers to the alleviation of a disease or disorder, and includes any action that ameliorates or beneficially alters the symptoms of a disease by inhibiting or reducing the progression of the disease or one or more clinical symptoms thereof.

In an embodiment, the daily dosage of the peptide or salt thereof may be 0.0002 to 100 mg/kg (body weight). Specifically, the daily dosage of the peptide or salt thereof may be 0.0002 mg/kg (body weight) or more, 0.0003 mg/kg (body weight) or more, 0.0005 mg/kg (body weight) or more, 0.0007 mg/kg (body weight) or more, 0.001 mg/kg (body weight) or more, 0.005 mg/kg (body weight) or more, 0.01 mg/kg (body weight) or more, 0.03 mg/kg (body weight) or more, 0.05 mg/kg (body weight) or more, 0.07 mg/kg (body weight) or more, 0.08 mg/kg (body weight) or more, 0.1 mg/kg (body weight) or more, 0.5 mg/kg (body weight) or more, 1 mg/kg (body weight) or more, 5 mg/kg (body weight) or more, 10 mg/kg (body weight) or more, 15 mg/kg (body weight) or more, 20 mg/kg (body weight) or more, 25 mg/kg (body weight) or more, 30 mg/kg (body weight) or more, 35 mg/kg (body weight) or more, 40 mg/kg (body weight) or more, 45 mg/kg (body weight) or more, 50 mg/kg (body weight) or more, 60 mg/kg (body weight) or more, 70 mg/kg (body weight) or more, 80 mg/kg (body weight) or more, or 90 mg/kg (body weight) or more, and in addition, the daily dosage of the peptide or salt thereof may be 100 mg/kg (body weight) or less, 90 mg/kg (body weight) or less, 80 mg/kg (body weight) or less, 60 mg/kg (body weight) or less, 50 mg/kg (body weight) or less, 45 mg/kg (body weight) or less, 40 mg/kg (body weight) or less, 35 mg/kg (body weight) or less, 30 mg/kg (body weight) or less, 25 mg/kg (body weight) or less, 20 mg/kg (body weight) or less, 15 mg/kg (body weight) or less, 10 mg/kg (body weight) or less, 5 mg/kg (body weight) or less, 1 mg/kg (body weight) or less, 0.9 mg/kg (body weight) or less, 0.5 mg/kg (body weight) or less, 0.1 mg/kg (body weight) or less, 0.07 mg/kg (body weight) or less, 0.05 mg/kg (body weight) or less, 0.03 mg/kg (body weight) or less, 0.01 mg/kg (body weight) or less, 0.005 mg/kg (body weight) or less, 0.001 mg/kg (body weight) or less, 0.0007 mg/kg (body weight) or less, or 0.0005 mg/kg (body weight) or less.

In an embodiment, the peptide or salt thereof may be included in the pharmaceutical composition in a therapeutically effective amount, and may further include a pharmaceutically acceptable carrier. The term “therapeutically effective amount” refers to an amount sufficient to achieve the purpose of the present composition, which is to prevent or treat alopecia.

In the present specification, the hair loss refers to the absence of hair in a region where hair should be normally present, and includes a phenomenon in which the hair count is reduced compared to the normal state. The hair loss is a concept that includes both non-scarring hair loss and scarring hair loss depending on whether hair follicles are destroyed, and examples thereof include all of the hereditary androgenic alopecia, alopecia areata, tinea capitis caused by fungal infection, telogen effluvium, trichotillomania, hair production disorder, alopecia caused by lupus, folliculitis decalvans, lichen planopilaris, hair loss caused by burns and/or trauma, and the like.

The hair loss may be prevented or treated by eliminating the cause of the hair loss as described above or suppressing the progression of hair loss, and hair growth may be promoted by suppressing hair from falling out or promoting hair formation.

In an embodiment, the pharmaceutical composition may be prepared in the form of a unit-dose or by being contained in a multi-dose container by being formulated using a pharmaceutically acceptable carrier and/or excipient according to a method that can be readily implemented by a person with ordinary skill in the art to which the present invention pertains. In this case, a formulation may also be in the form of a solution in an oil or aqueous medium, a suspension or in the form of an emulsion, an extract, a powder, a granule, a tablet, a capsule or a gel (for example, hydrogel), and the pharmaceutical composition may additionally include a dispersant or a stabilizer.

In an embodiment, the peptide or salt thereof, which the pharmaceutical composition includes, may be carried in a pharmaceutically acceptable carrier, such as a colloidal suspension, a powder, a saline solution, a lipid, a liposome, a microsphere, or a nanospherical particle. These may be complexed or associated with delivery vehicles, and may be transported in vivo using delivery systems known in the art, such as lipids, liposomes, microparticles, gold, nanoparticles, polymers, condensation reagents, polysaccharides, polyamino acids, dendrimers, saponins, adsorption enhancers or fatty acids.

In addition to them, the pharmaceutically acceptable carrier may include lactose, dextrose, sucrose, sorbitol, mannitol, starch, acacia, rubber, calcium phosphate, alginate, gelatin, calcium silicate, microcrystalline cellulose, polyvinylpyrrolidone, cellulose, water, syrup, methylcellulose, methyl hydroxybenzoate, propyl hydroxybenzoate, talc, magnesium stearate, mineral oil, and the like, which are typically used during formulation, but is not limited thereto. Furthermore, the pharmaceutically acceptable carrier may further include a lubricant, a wetting agent, a sweetener, a flavoring agent, an emulsifier, a suspending agent, a preservative, and the like in addition to the above ingredients.

In an embodiment, the pharmaceutical composition can be orally or parenterally administered, and may be used in the form of a general medicinal preparation. That is, the pharmaceutical composition of the present invention may be administered as various oral and parenteral formulations, and during the preparation, the pharmaceutical composition of the present invention is prepared by using a diluent or an excipient, such as a filler, an extender, a binder, a wetting agent, a disintegrant, and a surfactant. A solid preparation for oral administration includes a tablet, a pill, a powder, granules, a capsule, and the like, and the solid preparation is formulated by mixing at least one excipient, for example, starch, calcium carbonate, sucrose, lactose, gelatin, and the like with a herbal extract or fermented herbal product. Further, in addition to a simple excipient, lubricants such as magnesium stearate and talc are also used. A liquid preparation for oral administration corresponds to a suspension agent, a liquid for internal use, an emulsion, a syrup, and the like, and the liquid preparation may include various excipients, for example, a wetting agent, a sweetener, an aroma, a preservative, and the like, in addition to water and liquid paraffin which are commonly used simple diluents. Examples of a formulation for parenteral administration include a sterilized aqueous solution, a non-aqueous solvent, a suspension, an emulsion, a freeze-dried preparation, and a suppository. As the non-aqueous solvent and the suspension solvent, it is possible to use propylene glycol, polyethylene glycol, a vegetable oil such as olive oil, an injectable ester such as ethyl oleate, and the like. As a base of the suppository, it is possible to use Witepsol, Macrogol, Tween 61, cacao butter, laurin fat, glycerol, gelatin, and the like.

In an embodiment, the pharmaceutical composition may be an external skin preparation. The external skin preparation is a preparation which can be used by application to the outside of the skin, and when the pharmaceutical composition of the present invention is used as an external skin preparation, it may be applied to the scalp, specifically the scalp in an area where alopecia has occurred or the scalp in an area where hair growth is to be promoted. The external skin preparation may be a cream, a gel, an ointment, a skin emulsion, a skin suspension, a transdermal patch, a drug-containing bandage, a lotion, or a combination thereof. The external skin preparation may be appropriately compounded into an ingredient typically used in skin external preparations such as cosmetics or pharmaceuticals, such as an aqueous component, an oil-based component, a powder component, alcohols, a moisturizer, a thickener, an ultraviolet absorber, a whitening agent, a preservative, an antioxidant, a surfactant, a fragrance, a coloring agent, various skin nutrients, or a combination thereof, if necessary. Into the external skin preparation may also appropriately be compounded metal sequestering agents such as disodium edetate, trisodium edetate, sodium citrate, sodium polyphosphate, sodium metaphosphate, and gluconic acid, drugs such as caffeine, tannin, verapamil, licorice extract, glabridin, extract of Chinese quince fruit with hot water, various herbal medicines, tocopherol acetate, glycyrrhizic acid, tranexamic acid and derivatives or salts thereof, vitamin C, magnesium ascorbate phosphate, ascorbic acid glucoside, arbutin, kojic acid, saccharides such as glucose, fructose, and trehalose, and the like.

In an embodiment, the composition may be a cosmetic composition.

In one embodiment, the daily application amount of the peptide or salt thereof may be 0.0002 to 100 mg/kg (body weight). Specifically, the daily application amount of the peptide or salt thereof may be 0.0002 mg/kg (body weight) or more, 0.0003 mg/kg (body weight) or more, 0.0005 mg/kg (body weight) or more, 0.0007 mg/kg (body weight) or more, 0.001 mg/kg (body weight) or more, 0.005 mg/kg (body weight) or more, 0.01 mg/kg (body weight) or more, 0.03 mg/kg (body weight) or more, 0.05 mg/kg (body weight) or more, 0.07 mg/kg (body weight) or more, 0.08 mg/kg (body weight) or more, 0.1 mg/kg (body weight) or more, 0.5 mg/kg (body weight) or more, 1 mg/kg (body weight) or more, 5 mg/kg (body weight) or more, 10 mg/kg (body weight) or more, 15 mg/kg (body weight) or more, 20 mg/kg (body weight) or more, 25 mg/kg (body weight) or more, 30 mg/kg (body weight) or more, 35 mg/kg (body weight) or more, 40 mg/kg (body weight) or more, 45 mg/kg (body weight) or more, 50 mg/kg (body weight) or more, 60 mg/kg (body weight) or more, 70 mg/kg (body weight) or more, 80 mg/kg (body weight) or more, or 90 mg/kg (body weight) or more, and in addition, the daily application amount of the peptide or salt thereof may be 100 mg/kg (body weight) or less, 90 mg/kg (body weight) or less, 80 mg/kg (body weight) or less, 60 mg/kg (body weight) or less, 50 mg/kg (body weight) or less, 45 mg/kg (body weight) or less, 40 mg/kg (body weight) or less, 35 mg/kg (body weight) or less, 30 mg/kg (body weight) or less, 25 mg/kg (body weight) or less, 20 mg/kg (body weight) or less, 15 mg/kg (body weight) or less, 10 mg/kg (body weight) or less, 5 mg/kg (body weight) or less, 1 mg/kg (body weight) or less, 0.9 mg/kg (body weight) or less, 0.5 mg/kg (body weight) or less, 0.1 mg/kg (body weight) or less, 0.07 mg/kg (body weight) or less, 0.05 mg/kg (body weight) or less, 0.03 mg/kg (body weight) or less, 0.01 mg/kg (body weight) or less, 0.005 mg/kg (body weight) or less, 0.001 mg/kg (body weight) or less, 0.0007 mg/kg (body weight) or less, or 0.0005 mg/kg (body weight) or less.

In an embodiment, the cosmetic composition may include a cosmetically effective amount of the peptide or salt thereof and a cosmetically acceptable carrier, and the cosmetically effective amount refers to an amount sufficient to achieve the above-described alopecia prevention or hair growth promoting efficacy.

In an embodiment, the cosmetic composition may additionally contain other ingredients that have characteristics that can provide, for example, a synergistic effect to the activity of the peptide within a range that does not affect the hair loss prevention or hair growth promotion activity of the peptide. For example, the cosmetic composition may contain adjuvants typically used in the cosmetic or dermatological field, such as a fatty substance, an organic solvent, a solubilizing agent, a thickening agent and a gelling agent, a softener, an antioxidant, a suspending agent, a stabilizer, a foaming agent, an aroma, a surfactant, water, an ionic or a non-ionic emulsifier, a filler, a metal ion sequestering agent, a chelating agent, a preservative, a vitamin, a blocking agent, a wetting agent, an essential oil, a dye, a pigment, a fragrance, a hydrophilic or lipophilic active agent, a lipid vesicle, or any other ingredient typically used in cosmetics, and the ingredients may be contained in amounts generally used in the cosmetic or dermatological field.

In an embodiment, the cosmetic composition may a hair tonic, a hair conditioner, a hair essence, a hair lotion, a hair nourishing lotion, a hair shampoo, a hair rinse, a hair treatment, a hair cream, a hair nourishing cream, a hair moisturizing cream, a hair massage cream, a hair wax, a hair aerosol, a hair pack, a hair nourishing pack, a hair soap, a hair cleansing foam, a hair oil, a hair drying agent, a hair preservation treatment agent, a hair colorant, a hair waving preparation, a hair decolorant, a hair gel, a hair glaze, a hair dressing agent, a hair lacquer, a hair moisturizer, a hair mousse, a hair spray, an eyebrow growth agent, an eyelash growth agent, or an eyelash nutrition agent.

In an embodiment, the cosmetic composition may be prepared in any formulation typically prepared in the art, and may be formulated into, for example, a solution, a suspension, an emulsion, a paste, a powder, a gel, a cream, a lotion, a soap, an oil, a spray, and the like, but is not limited thereto.

When the formulation of the cosmetic composition is a paste, a cream or a gel, an animal oil, a vegetable oil, a wax, paraffin, starch, tragacanth, a cellulose derivative, a polyethylene glycol, silicone, bentonite, silica, talc, zinc oxide, or the like may be used as a carrier ingredient.

When the formulation of the cosmetic preparation is a powder or a spray, lactose, talc, silica, aluminum hydroxide, calcium silicate, or a polyamide powder may be used as the carrier ingredient, and in particular, when the formulation of the present invention is a spray, the formulation may additionally include a propellant such as a chlorofluorohydrocarbon, propane/butane or dimethyl ether.

When the formulation of the cosmetic composition is a solution or an emulsion, a solvent, a solubilizer or an emulsifier is used as the carrier ingredient, and examples thereof include water, ethanol, isopropanol, ethyl carbonate, ethyl acetate, benzyl alcohol, benzyl benzoate, propylene glycol, 1,3-butylglycol oil, glycerol aliphatic esters, polyethylene glycol or fatty acid esters of sorbitan.

When the formulation of the cosmetic composition is a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used as a carrier ingredient.

In addition to the active ingredients and carrier ingredients, the ingredient included in the cosmetic composition may include ingredients that are typically used in external skin preparation compositions, and may include a typical adjuvant, such as, for example, an antioxidant, a stabilizer, a solubilizer, a vitamin, a pigment, and a fragrance.

In an embodiment, the composition may be a food composition, a health functional food composition, or a non-therapeutic oral composition.

In an embodiment, the daily intake of the peptide or salt thereof may be 0.0002 to 100 mg/kg (body weight). Specifically, the daily intake of the peptide or salt thereof may be 0.0002 mg/kg (body weight) or more, 0.0003 mg/kg (body weight) or more, 0.0005 mg/kg (body weight) or more, 0.0007 mg/kg (body weight) or more, 0.001 mg/kg (body weight) or more, 0.005 mg/kg (body weight) or more, 0.01 mg/kg (body weight) or more, 0.03 mg/kg (body weight) or more, 0.05 mg/kg (body weight) or more, 0.07 mg/kg (body weight) or more, 0.08 mg/kg (body weight) or more, 0.1 mg/kg (body weight) or more, 0.5 mg/kg (body weight) or more, 1 mg/kg (body weight) or more, 5 mg/kg (body weight) or more, 10 mg/kg (body weight) or more, 15 mg/kg (body weight) or more, 20 mg/kg (body weight) or more, 25 mg/kg (body weight) or more, 30 mg/kg (body weight) or more, 35 mg/kg (body weight) or more, 40 mg/kg (body weight) or more, 45 mg/kg (body weight) or more, 50 mg/kg (body weight) or more, 60 mg/kg (body weight) or more, 70 mg/kg (body weight) or more, 80 mg/kg (body weight) or more, or 90 mg/kg (body weight) or more, and in addition, the daily intake of the peptide or salt thereof may be 100 mg/kg (body weight) or less, 90 mg/kg (body weight) or less, 80 mg/kg (body weight) or less, 60 mg/kg (body weight) or less, 50 mg/kg (body weight) or less, 45 mg/kg (body weight) or less, 40 mg/kg (body weight) or less, 35 mg/kg (body weight) or less, 30 mg/kg (body weight) or less, 25 mg/kg (body weight) or less, 20 mg/kg (body weight) or less, 15 mg/kg (body weight) or less, 10 mg/kg (body weight) or less, 5 mg/kg (body weight) or less, 1 mg/kg (body weight) or less, 0.5 mg/kg (body weight) or less, 0.1 mg/kg (body weight) or less, 0.09 mg/kg (body weight) or less, 0.07 mg/kg (body weight) or less, 0.05 mg/kg (body weight) or less, 0.03 mg/kg (body weight) or less, 0.01 mg/kg (body weight) or less, 0.005 mg/kg (body weight) or less, 0.001 mg/kg (body weight) or less, 0.0007 mg/kg (body weight) or less, or 0.0005 mg/kg (body weight) or less.

In an embodiment, a formulation of the food composition or non-therapeutic oral composition is not particularly limited, but the food composition or non-therapeutic oral composition may be formulated into, for example, a tablet, a granule, a pill, a powder, a liquid such as a drink, a caramel, a gel, a bar, a tea bag, or the like. For the composition of each formulation, ingredients typically used in the art may be appropriately selected and compounded by a person with ordinary skill in the art according to the dosage form or purpose of use, in addition to the active ingredient, and when the composition is simultaneously applied with other raw materials, a synergistic effect may occur.

In an embodiment, the food composition or non-therapeutic oral composition may be administered by various methods such as simple ingestion, drinking, injection administration, spray administration, or squeeze administration.

In an embodiment, the food composition or non-therapeutic oral composition may be, for example, various foods such as chewing gum, chocolate, caramel products, candies, frozen treats, and confectionery, beverage products such as soft drinks, mineral water, and alcoholic beverages, and health functional foods including vitamins or minerals.

In an embodiment, the food composition or non-therapeutic oral composition may be taken as is or used together with other foods or food ingredients, and may be appropriately used by a typical method. The food composition or non-therapeutic oral composition may include a sitologically acceptable food supplement additive in addition to the peptide or salt thereof, and the amount of the active ingredient mixed may be appropriately determined depending on the purpose of use.

As used herein, the “food supplement additive” refers to a component that can be added to food as a supplement, and is added to prepare a health functional food of each formulation, and may be appropriately selected and used by those skilled in the art. Examples of the food supplement additive include various nutrients, vitamins, minerals (electrolytes), flavoring agents such as synthetic flavoring agents and natural flavoring agents, colorants and fillers, pectic acid and salts thereof, alginic acid and salts thereof, organic acids, protective colloid thickeners, pH adjusting agents, stabilizers, preservatives, glycerin, alcohols, carbonating agents used in carbonated beverages, and the like, but the types of food supplement additives of the present invention are not limited by the above examples.

As used herein, the “health functional food” refers to a food prepared and processed in the form of a tablet, capsule, powder, granule, liquid, pill, and the like using raw materials or ingredients that have functionality useful to the human body. Here, ‘functionality’ means that useful effects for health applications such as regulating nutrients and physiological actions are obtained for the structure and function of the human body. The health functional food of the present invention can be prepared by a method typically used in the art, and may be prepared by adding raw materials and ingredients typically added in the art during preparation. In addition, the formulation of the health functional food may also be prepared without limitation as long as it is recognized as a health functional food. The food composition may be prepared in various forms of formulations.

The present invention may provide the following embodiments as an example.

The first embodiment may provide a method for suppressing hair loss or promoting hair growth, the method comprising: administering an effective amount of a peptide including an amino acid sequence of Gly-Pro-Lys (GPK), an amino acid sequence of Gly-Pro-Ser (GPS), an amino acid sequence of Gly-Pro-Ala (GPA), an amino acid sequence of Gly-Gln-Lys (GQK), or an amino acid sequence of Pro-Hyp (PO); or a salt thereof to a subject in need thereof.

The second embodiment may provide the method of the first embodiment, wherein the peptide is a GPK, GPS, GPA, GQK, or PO peptide.

The third embodiment may provide the method of one or more of the first embodiment and the second embodiment, wherein the peptide or salt thereof is in the form of a composition, and is included in the composition at a concentration of 0.1 to 100 μM.

The fourth embodiment may provide the method of one or more of the first embodiment to the third embodiment, wherein cells are treated with the peptide or salt thereof in the composition at a concentration of 0.1 to 10 μM.

The fifth embodiment may provide the method of one or more of the first embodiment to the fourth embodiment, wherein the cells are hair dermal papilla cells.

The sixth embodiment may provide the method of one or more of the first embodiment to the fifth embodiment, wherein the peptide or salt thereof promotes or activates the growth or proliferation of hair dermal papilla cells.

The seventh embodiment may provide the method of one or more of the first embodiment to the sixth embodiment, wherein the peptide or salt thereof is formulated into the form of a pharmaceutical composition for preventing or treating hair loss.

The eighth embodiment may provide the method of one or more of the first embodiment to the seventh embodiment, wherein the daily dosage of the peptide or salt thereof is 0.0003 to 30 mg/kg (body weight).

The ninth embodiment may provide the method of one or more of the first embodiment to the eighth embodiment, wherein the peptide or salt thereof is formulated into the form of a cosmetic composition.

The tenth embodiment may provide the method of one or more of the first embodiment to the ninth embodiment, wherein the daily application amount of the peptide or salt thereof is 0.0003 to 30 mg/kg (body weight).

The eleventh embodiment may provide the method of one or more of the first embodiment to the tenth embodiment, wherein the peptide or salt thereof is formulated into the form of a food composition.

The twelfth embodiment may provide the method of one or more of the first embodiment to the eleventh embodiment, wherein the daily intake of the peptide or salt thereof is 0.0003 to 30 mg/kg (body weight).

Hereinafter, the contents of the present invention will be described in more detail through examples. However, these examples are presented merely for the purpose of understanding the contents of the present invention, and the scope of the present invention is not limited to these examples, modifications, substitutions, insertions, and the like that are typically known in the art may be made, and such modifications are also included in the scope of the present invention.

### Example: Confirmation of the Effect of Activating Hair Dermal Papilla Cell Proliferation

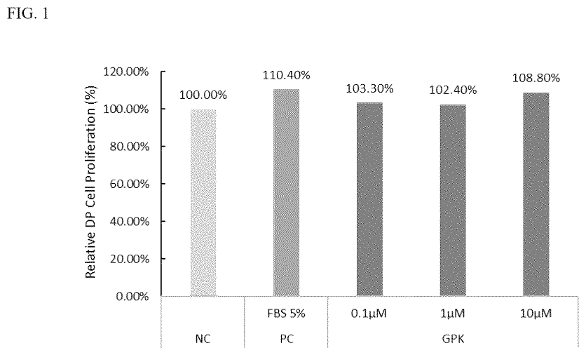

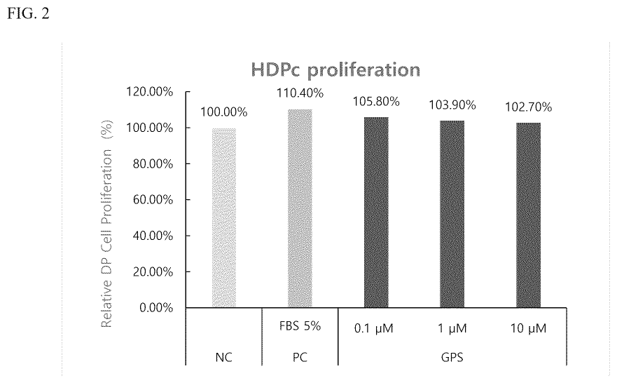

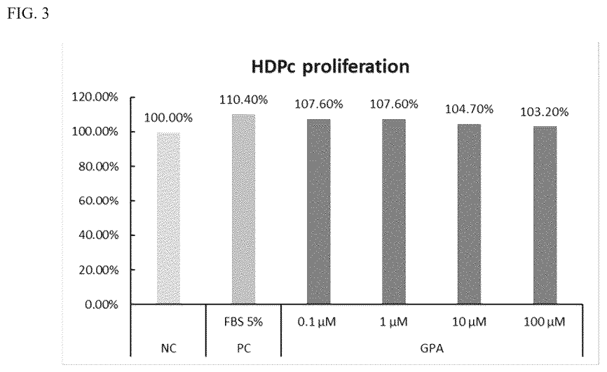

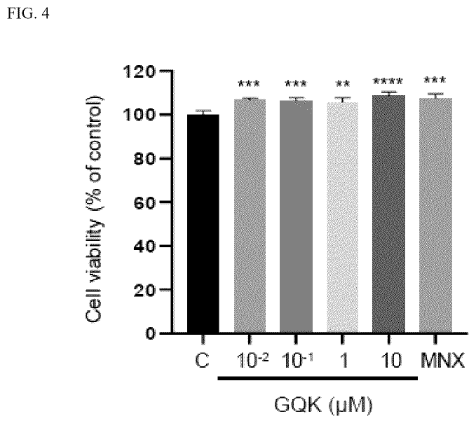

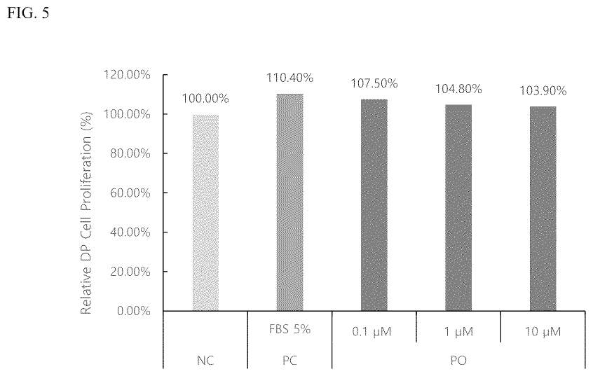

Human dermal papilla cells (HDPCs) were attached to a 96-well plate at 2,000 cells per well, and then cultured using DMEM medium (Sigma Aldrich) containing 10% FBS and 1% penicillin/streptomycin (P/S; Sigma Aldrich) for 24 hours, the medium was then exchanged with serum-free DMEM, and diluted samples were treated with a GPK, GPS, GPA, GQK, or PO peptide at a concentration of each of 0.1 μM, 1 μM, and 10 μM, for 72 hours, and then treated with a CCK8 reagent for 1 hour to measure the absorbance at 450 nm using a microplate reader (TECAN), and the results are shown in FIGS. 1 to 5 (FIG. 1: GPK, FIG. 2: GPS, FIG. 3: GPA, FIG. 4: GQK, and FIG. 5: PO). As a positive control (PC), a 5% fetal bovine serum (FBS) treated group was used.

From the results of FIGS. 1 to 5, it could be confirmed that when GPK, GPS, GPA, GQK or PO peptide was treated at a concentration of 0.1 μM, 1 μM and 10 μM, cell proliferation was significantly increased compared to the untreated control, and in particular, when treated at a concentration of 10 μM, the effect of activating hair dermal papilla cell proliferation was excellent, at a level similar to that of the positive control.

We claim:

1. A method for suppressing hair loss or promoting hair growth, the method comprising: administering an effective amount of a peptide comprising an amino acid sequence of Gly-Pro-Lys (GPK), an amino acid sequence of Gly-Pro-Ser (GPS), an amino acid sequence of Gly-Pro-Ala (GPA), an amino acid sequence of Gly-Gln-Lys (GQK), or an amino acid sequence of Pro-Hyp (PO); or a salt thereof to a subject in need thereof.

2. The method of claim 1, wherein the peptide is a GPK, GPS, GPA, GQK, or PO peptide.

3. The method of claim 1, wherein the peptide or salt thereof is formulated into a form of a composition, and is comprised in the composition at a concentration of 0.001 to 100 μM.

4. The method of claim 1, wherein cells are treated with the peptide or salt thereof at a concentration of 0.1 to 10 μM.

5. The method of claim 4, wherein the cells are hair dermal papilla cells.

6. The method of claim 1, wherein the peptide or salt thereof promotes or activates the growth or proliferation of hair dermal papilla cells.

7. The method of claim 1, wherein the peptide or salt thereof is formulated into a form of a pharmaceutical composition for preventing or treating hair loss.

8. The method of claim 7, wherein a daily dosage of the peptide or salt thereof is 0.0003 to 30 mg/kg (body weight).

9. The method of claim 1, wherein the peptide or salt thereof is formulated into a form of a cosmetic composition.

10. The method of claim 9, wherein a daily application amount of the peptide or salt thereof is 0.0003 to 30 mg/kg (body weight).

11. The method of claim 1, wherein the peptide or salt thereof is formulated into a form of a food composition.

12. The method of claim 11, wherein a daily intake of the peptide or salt thereof is 0.0003 to 30 mg/kg (body weight).
